(19) United States
(12) Patent Application Publication
(10) Pub. No.: US 2006/0275279 A1
(43) Pub. Date: Dec. 7, 2006

(54) Methods for dissolving cystine stones and reducing cystine in urine

(76) Inventors: J. David Rozzell, Burbank, CA (US); Kavitha Vedha-Peters, Pasadena, CA (US)

Correspondence Address: CHRISTIE, PARKER & HALE, LLP, PO BOX 7068, PASADENA, CA, 91109-7068, US

(21) Appl. No.: 11/146,551

(22) Filed: Jun. 6, 2005

(51) Int. Cl. A61K 38/51

(52) U.S. Cl. 424/94.5

(57) ABSTRACT

The present invention is directed to an improved method of treating cystinuria, utilizing the catalytic ability of cystinase to increase the rate of cystine stone dissolution.

### BACKGROUND

The cause of cystinuria, a genetic disease, is well understood. In healthy individuals, cystine is filtered from blood at the renal glomeruli and reabsorbed by the proximal renal tubule cells via a transporter protein assembly that is specialized for certain amino acids including cystine, arginine, lysine, ornithine and citrulline. In cystinurics this amino acid transporter assembly is defective, and cystine is not reabsorbed normally. The cystine accumulates in the urine in abnormally large amounts and, due to its insolubility relative to other amino acids, crystallizes to produce stones.

Cystinuria causes one of the most dangerous types of kidney stones, and cystinurics often experience a life of misery due to frequent stone formation episodes. Cystine stones are far more serious than the common calcium oxalate stone because they can be over twenty-times larger than oxalate stones in weight and size. Cystine stone formers experience severe pain and frequently require emergency room visits, hospitalizations, and surgeries. Current treatment regimens for cystine stones are often difficult and unsuccessful. As a result, renal failure that results in a need for dialysis or kidney transplantation is not uncommon. Since no highly effective treatments for cystine stones exist, cystinurics face extremely difficult living circumstances, and the costs of cystinuria to the medical system are high, despite the low patient population (Beaudet, 1995).

Given the severity of the symptoms of cystinuria, treatment is essential. The first course of treatment usually involves management of urinary cystine levels to reduce the risk of stone formation. These management methods include substantially increasing the intake of water (thereby increasing the urine volume and the amount of cystine that can be solubilized), dietary restrictions of methionine, which is a metabolic precursor of cystine, and sodium, and oral administration of potassium citrate to increase the pH of the urine, thereby increasing the solubility of cystine. When these methods are ineffective, drug therapy is often used.

Drug therapy involves the use of thiol-containing drugs, such as D-penicillamine, α-mercaptopropionylglycine (Thiola), and captopril, to break the cystine disulfide bond and form more soluble mixed disulfides. However, these drugs frequently give the patient various unpleasant side effects such as gastrointestinal intolerance, rash and pain in the joints (Sakhaee and Sutton, 1996). These management techniques for cystinuric patients are often not successful; one study saw 14 of 16 patients, who were using these management methods, nevertheless develop cystine stones (Chow and Streem, 1996).

When cystine stone formation cannot be prevented using hydration, dietary restrictions and drug therapy, surgical management is necessary. Cystinuric patients often have recurrent episodes of stone formation and surgeries in their lifetime. Shock wave lithotripsy, the use of high-energy shock waves for stone fragmentation, can be used for treatment of cystine stones that are smaller than 1.5 cm. Cystine stones are the most sturdy of all urinary stones and lithotripsy is generally ineffective in breaking them up. However, smaller cystine stones may be fragmented with lithotripsy because more frequent shocks at higher energy can be used.

One of the most common methods for the removal of cystine stones is percutaneous nephrolithotomy, in which a keyhole incision is made in the back and a nephroscope is used to break up and remove the stones. Although this procedure is less invasive than open surgery, regular or spinal anesthesia is normally required along with a hospital stay of 2 to 3 days and a recovery time of a few weeks, in which the patient may not be able to work (Ng and Streem, 2001).

An additional method for the treatment of cystine stones, which is a non-surgical and minimally invasive route, involves the delivery of chemical solutions to the kidneys via a nephrostomy catheter for the chemical dissolution of the stones, also known as chemodissolution. A variety of chemolytic agents have been used in this technique including sodium bicarbonate and the organic buffer tris-hydroxymethylene-aminomethane (tromethamine-E) at pH 10, both which act to provide a strongly alkaline environment to dissolve the cystine stones. Acetylcysteine is also frequently used in chemodissolution and dissolves the stones in a manner similar to D-penicillamine and Thiola by breaking the cystine disulfide and forming more soluble disulfides. However, this dissolution method has a limited role in the treatment of cystine stones because these chemolytic agents perform extremely slowly and can typically take weeks to months to dissolve stones (Ng and Streem, 2001).

Given the drawbacks of the current methods for the treatment of cystine stones, a minimally invasive, non-surgical route for treatment in which the cystinuric is treated under general anesthesia and in outpatient care would be highly desirable. Chemodissolution possess these desirable characteristics, however the slow rate of dissolution using the current chemical solutions makes this an unfeasible treatment.

The present invention overcomes this major drawback of chemodissolution by utilizing the catalytic ability of an enzyme to increase the rate of cystine stone dissolution to provide an improved method of treatment.

### SUMMARY OF THE INVENTION

One embodiment of the present invention is directed to a method for dissolving cystine stones, comprising the step of administering to a patient in need thereof a therapeutically effective amount of a cystinase.

Another embodiment of the present invention is directed to a method for the treatment of cystinuria, comprising administering to a patient in need thereof a therapeutically effective amount of a cystinase.

Another embodiment of the present invention is directed to a method for lowering the concentration of cystine in a patient's urine, comprising the step of administering a therapeutically effective amount of a cystinase to the patient.

In one embodiment of the present invention, the cystinase is cystathionine β-lyase.

In another embodiment of the present invention, the cystinase is selected from the group consisting of cystothionine-gamma-lyase, cystine transaminase, cystine transhydrogenase, cystine reductase, cystathionine β-lyase, and mixtures thereof.

In another embodiment of the present invention, the cystinase is covalently attached to a polymer. In one embodiment, that polymer is polyethylene glycol.

In still another embodiment of the present invention, the cystinase is entrapped in a polymeric material. In one embodiment, that polymeric material comprises at least about 50% of a polylalkanoic acid, optionally polylactic acid.

In another embodiment of the present invention, the cystinase is administered via a catheter into the kidney of the patient.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 is a schematic drawing showing the action of the cystinase enzyme cystathionine β-lyase, isolated from E. coli bacteria, in catalyzing the degradation of L-cystathionine to L-homocysteine in the biosynthesis of L-methionine.

FIG. 2 is a schematic drawing showing the conversion of L-cystine into pyruvate, ammonia and thiocysteine, catalyzed by cystathionine β-lyase.

FIG. 3. Graph showing the rate of dissolution of cystine stones by the cystinase cystathionine β-lyase.

FIG. 4 Scans of a 6-well plate showing dissolution of cystine stones at various time points.

### DETAILED DESCRIPTION OF THE INVENTION

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing, for example, the cell lines, constructs, and methodologies that are described in the publications which might be used in connection with the presently described invention. The publications discussed above, below and throughout the text are provided solely for their disclosure prior to the filing date of the present application. Nothing herein is to be construed as an admission that the inventors are not entitled to antedate such disclosure by virtue of prior invention.

The present invention is directed to an improved method of treating cystinuria, utilizing the catalytic ability of an enzyme, such as a cystinase, to increase the rate of cystine stone dissolution. As used herein, “cystinase” refers to any enzyme that catalyzes a chemical change in the amino acid cystine to increase the solubility thereof.

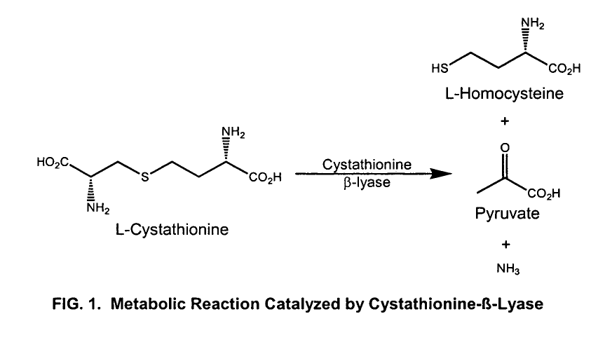

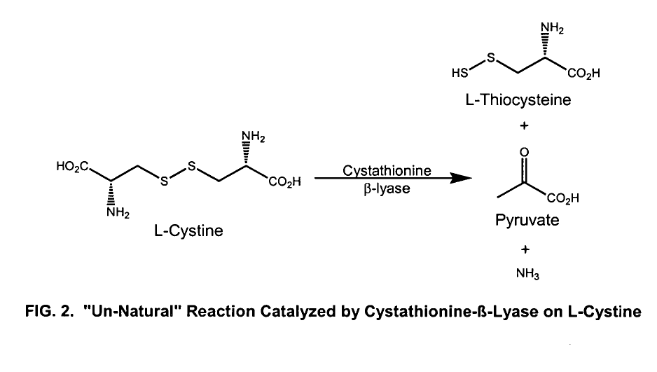

One such cystinase is the enzyme cystathionine β-lyase, which has been isolated from E. coli bacteria and catalyzes as its natural reaction the degradation of L-cystathionine to L-homocysteine in the biosynthesis of L-methionine (FIG. 1). This same enzyme has also been reported to catalyze at a comparable rate an “un-natural” reaction: the conversion of L-cystine into pyruvate, ammonia and thiocysteine, as shown in FIG. 2 (Dwivedi et al., 1982). Other cystinases include cystothionine-gamma-lyase, cystine transaminase, cystine transhydrogenase, and cystine reductase. Others will be apparent to those of skill in the art.

Because of the high activity of cystinases toward cystine and the production of soluble compounds upon its breakdown, these enzymes can be utilized for the dissolution of cystine stones in the kidneys through the use of a nephrostomy catheter in a manner similar to the chemodissolution method currently used. Thus, one embodiment of the present invention is directed to the use of a cystinase for dissolution of cystine stones in the kidneys.

General Materials and Methods. The pET15b expression vector and the expression host E. coli BL21(DE3) cells were purchased from Novagen (Madison, Wis.) and Invitrogen (Carlsbad, Calif.), respectively. The monomethoxy-polyethylene glycol-succinimidyl propionate (mPEG-SPA) for use in the pegylation is available from Nektar Therapeutics (Huntsville, Ala.). Other key materials are available from well-known vendors such as Sigma-Aldrich Company (St. Louis, Mo.), VWR (Pittsburgh, Pa.), and the like.

Enzyme Production and Purification. E. coli strain GI724 containing the metC gene is cultivated in 2 liter shaker flasks containing 400 ml of RM base media containing 0.5% glucose and 0.1 mg/ml ampicillin at 30° C. Growth is monitored by following the OD₆₀₀ of the culture. Once mid-logarithmic phase is achieved, induction may be initiated by the addition of 0.1 mM L-tryptophan and raising the temperature of the culture to 37° C. Approximately 4 hours after the initiation of induction, the cells are harvested by centrifugation at 13,000×g and washed with 50 mM potassium phosphate buffer, pH 7.0, containing 0.1 mM dithiothreitol and 0.05 mM pyridoxal phosphate. Cells not processed immediately may be stored in the presence of 15% glycerol at −80° C. until needed.

The metC gene product, cystathionine β-lyase, is isolated by cell lysis and removal of the cell debris by centrifugation, and then assayed using a modification of the method of Uren. (Uren, 1987) The rate of breakdown of cystathionine (the natural substrate for the enzyme) can be monitored by reaction of the homocysteine liberated with 5,5′-dithiobis(2-nitrobenzoic acid) (Ellman's reagent) to produce a yellow-colored mercaptide. The release of 2-nitro-5-thiobenzoic acid can be observed by monitoring the absorbance at 412 nm in a spectrophotometer. Using the known molar extinction coefficient for the mercaptide of 13,200 M⁻¹cm⁻¹, the rate of formation of cysteine from cystathionine can be calculated. One unit of cystathionine β-lyase activity equals the formation of 1 micromole of mercaptide per minute at room temperature.

The assay mixture is prepared as follows: 0.2 ml of 10 mM cystathionine solution in 0.01 M HCl was added to 0.8 ml of 50 mM potassium phosphate buffer, pH 8.0, containing 0.05 mM pyridoxal phosphate and 1 mM Ellman's reagent. A background reading of the absorbance at 412 nm is made. Enzyme solution (10 microliters, appropriately diluted to produce a change of absorbance between 0.1 and 1.0 absorbance unit per minute) is added, and the rate of increase in the absorbance at 412 nm measured using a UV-Visible spectrophotometer. Using the known molar extinction coefficient for the mercaptide of 13,200 M⁻¹ cm⁻¹, the rate of formation of cysteine from cystathionine can be calculated.

Cells are broken by mixing the cell paste with two volumes of cold 50 mM potassium phosphate buffer, pH 8.0, containing 0.1 mM dithiothreitol and 0.05 mM pyridoxal phosphate (purification buffer), followed by sonication. The cell lysate is then centrifuged at 13,000×g to remove cell debris. The clarified enzyme solution is loaded onto a DEAE-sepharose fast flow column equilibrated with the purification buffer. After washing the column with purification buffer until no more protein elutes from the column, the enzyme is eluted off the column with a linear gradient of 0-0.5 M NaCl in purification buffer, fractions containing cystathionine β-lyase activity are pooled. The active fractions are dialyzed against purification buffer and then poured onto a Blue Sepharose column, followed by elution with a 0-0.5 M linear gradient of NaCl. Active fractions are once again pooled and dialyzed. The enzyme solution can be stored at 4° C. in the presence of 0.02% sodium azide. For long term storage, the enzyme solution can be lyophilized in the presence of various inert stabilizers (lactose, potassium chloride, etc.).

Assay Methods. Enzyme activity may be determined using an enzyme-coupled assay, which is a modification of the method described by Gentry-Weeks et al. (Gentry-Weeks et al., 1993). Cystathionine β-lyase catalyzes the breakdown of cystathionine (the natural substrate for the enzyme) and cystine, releasing pyruvate as a product in both reactions. The pyruvate liberated is quantified by reaction with L-lactate dehydrogenase (L-LDH) and NADH. The activity is determined by monitoring the decrease in NADH concentration by measuring the decrease in absorbance at 340 nm (ε=6.22 mM⁻¹ cm⁻¹). One unit of cystathionine β-lyase activity equals the oxidation of 1 micromole of NADH (equivalent to pyruvate formed and substrate hydrolyzed) per minute at room temperature. The assay may be carried as follows:

Buffer: 150 mM potassium phosphate pH 8.5 containing 0.02 mM PLP

L-LDH: 15 U/mL

NADH: 0.25 mM

Substrate: 2 mM cystathionine or 1 mM cystine

cystathionine β-lyase: enough to give approximately 0.1 ΔAbs/min

The enzyme is added to the assay mixture of buffer, NADH and L-LDH and left for approximately one minute in order to react any pyruvate that may be in the enzyme sample. The assay is then be initiated by the addition of substrate (cystathionine or cystine). Purified cystathionine β-lyase has an activity of approximately 20 units per milligram of lyophilized enzyme, when assayed toward cystathionine. As will be appreciated by the skilled artisan, this assay may be modified for other cystinases, depending on their particular mode of action.

The purified cystathionine β-lyase has a high selectivity for L-cystine in the presence of other amino acids and compounds typically found in urine. The enzyme is not significantly inhibited by pyruvate or other 2-ketoacids that might be present in urine. In addition to L-cystine, only its natural substrate, L-cystathionine, and two relatively obscure compounds (lanthionine and djenkolic acid) have been shown to react (Dwivedi et al., 1982). Since the normal levels of cystathionine in urine are extremely low, only the reaction with cystine is observed in urine from cystinurics, where cystine concentrations can exceed 500 mg/L (normal concentration is approximately 10-20 mg/L). Furthermore, the enzyme functions well in urine.

Pegylation. When introduced into the body, enzymes can have a short circulating half-life, be susceptible to proteolytic degradation, and induce an immunological response. The covalent attachment of PEG molecules to the enzyme (pegylation) allows these problems to be overcome by masking the enzyme surface and increasing the molecular size. In addition to overcoming the problems stated above, pegylated enzymes also display reduced toxicity and increased physical stability, thermostability and solubility.

Pegylation can result in reduced specific activity of the enzyme, but the beneficial therapeutic enhancements offset this disadvantage. Several pegylated enzymes have been FDA approved and are currently being marketed. PEG-adenosine deaminase (Adagen), developed by Enzon, is used in the treatment of severe combined immunodeficiency syndrome, which is the result of a deficiency of adenosine deaminase. Enzon has also introduced PEG-asparaginase (Oncaspar) for the treatment of acute lymphoblastic leukemia. Two competing pegylated drugs developed by Schering Plough and Hoffman-La Roche, PEG-interferonα2b (PegIntron) and PEG-interferonα2a (Pegasys), respectively, are used for the treatment of hepatitis C. Additionally, several other pegylated enzymes are undergoing the approval process, such as PEG-granulocyte colony stimulating factor (Amgen) and PEG-human growth hormone antagonist from Pharmacia (Harris and Chess, 2003).

Thus, in a preferred embodiment of the present invention, pegylation of the cystinase enzyme is used to ensure the successful delivery of the enzyme to the kidneys for dissolution. The pegylation of cystinase provides the enzyme the beneficial characteristics described above thus enhancing the efficacy of the peglyated enzyme in the dissolution of urinary cystine stones.

Pegylated derivatives of cystinases are prepared by reacting the ε-amino group of lysine residues in the cystinase to a succinimidyl-activated ester of monomethoxy-polyethylene glycol propionic acid (mPEG-SPA). The use of the monofunctional monomethoxy-PEG (mPEG) reduces the amount of inactive crosslinked protein aggregates that may be formed during the PEG-enzyme coupling procedure. The pegylation reaction is carried out at room temperature in potassium phosphate buffer, pH 8, for approximately 5 hours. The reaction is performed separately with mPEG-SPA molecular weight 5,000 Da and 20,000 Da and the molar ratio of cystinase to mPEG-SPA varied from 1:1 to 1:5 in order to determine the optimal pegylation conditions. The reaction is quenched by adjusting the pH to approximately 4.5 and the pegylated cystinase purified using ion-exchange chromatography.

The reaction mixture is loaded onto a cation exchange column; the unconjugated protein binds onto the resin while the PEG-cystinase washes through with the buffer (Bailon and Berthold, 1998). Alternatively, depending on the molecular weight of the mPEG-SPA and the number of PEGs attached to the protein, size-exclusion chromatography may also be used in purification.

PEG conjugation to cystinase is determined before and after purification by sodium dodecyl sulfate-polyacrylamide gel electrophoresis (SDS-PAGE) with the NuPage electrophoresis system (Invitrogen). The gel is run with PEG and protein molecular weight standards and stained specifically for both PEG-cystinase and free protein according to the procedure described by Kurfurst (Kurfurst, 1992). In order to specifically stain the pegylated protein and PEG, the gel is soaked in 20 mL of 0.1 M perchloric acid for 15 minutes followed by the addition of 5 mL of a 5% barium chloride solution and 2 mL of a 0.1 M iodine solution; the stained bands should appear within a few minutes. After the gel is fully stained, it is soaked in water for approximately 15 minutes. The gel may then be stained for protein using the Simply Blue Safe Stain (Invitrogen). Using separate protein and PEG molecular weight standards and staining solutions will allow for a more accurate molecular weight determination of the pegylated protein. Pegylated protein concentrations may be assessed using the standard Bradford Protein Assay Kit (Sigma).

The purified pegylated cystinase may be assayed for activity toward cystine as described above. In addition to linear mPEG, branched mPEG may also be used for protein coupling. The use of branched mPEG should increase the activity of the pegylated enzyme because the size of the PEG molecule is increased without increasing the number of attachment sites, thus reducing the possibility of inactivation of the protein due to attachment by an active site residue (Roberts et al., 2002).

In addition to pegylation, polymers comprising at least about 50% of a polylalkanoic acid have been used to entrap materials such as enzymes. Thus, cystinase may be entrapped using known methods in a polymer such as polylactic acid to create a cystinase composition that has increased stability and slow release characteristics.

In vitro dissolution tests. Cystinase and/or pegylated derivatives of cystinase may used to perform in vitro dissolution experiments with cystine stones, approximately 2-3 mm in diameter and from 5-15 mg in weight, that have been donated from a cystinuric patient. Initially to determine the optimal enzyme concentration and buffer conditions, the experiments are carried out in a 6 or 12-well plate with each well containing a cystine stone and varying enzyme concentration and buffer conditions. Every experimental condition includes a control well containing no enzyme in order to determine whether any background dissolution is occurring, especially when higher pH buffers are used. The cystine stones are weighed at the start of the experiment and then added to the wells containing 2-4 mL of the selected buffer containing varying amounts of enzyme (or pegylated enzyme). The plate is shaken at approximately 75 rpm at 37° C. and the weight of the stone monitored over time. The reactions may be carried out using reconstituted lyophilized human urine (Sigma) at pH 5.5 to 6, to mimic physiological conditions, and 25 mM potassium phosphate buffer at pH 8.5, which is the pH optimum for the enzyme.

The use of pH 8.5 buffer in the dissolution experiments will provide an advantage because cystine has increased solubility at a higher pH in addition to.resulting in optimal activity of the enzyme at this pH. Potassium phosphate buffer at a lower pH may also be used. The varying cystinase and/or PEG-cystinase concentrations that are used may be determined based on the activity determined from the spectrophotometric assays.

The conditions that provide the optimal cystine stone dissolution over time may also be used to perform dissolution experiments similar to studies described in the literature, that mimic the physiological conditions in the upper urinary tract (Heimbach et al., 2000). The cystine stone may be enclosed in a cell held constant at 37° C. with inlet and outlet tubing, where a solution of fresh cystinase (or pegylated cystinase) is added into this cell from a reservoir using a peristaltic pump. Fresh cystinase (or PEG-cystinase) is passed through the cell containing the cystine stone at a rate of approximately 80 mL/hr. The stone is weighed periodically to monitor the extent of dissolution. This experiment allows one to determine the amount of enzyme and length of time of exposure to the enzyme that is necessary to dissolve small cystine stones.

Therapeutic Agents. Cystinase can be formulated into pharmaceutical compositions by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. Administration of the compounds can be administered in a variety of ways known in the art, as, for example, by oral, buccal, rectal, parenteral, intraperitoneal, intradermal, transdermal, intratracheal, etc., administration.

Depending upon the particular route of administration, a variety of pharmaceutically acceptable carriers, well known in the art can be used. These carriers include, but are not limited to, sugars, starches, cellulose and its derivatives, malt, gelatin, talc, calcium sulfate, vegetable oils, synthetic oils, polyols, alginic acid, phosphate buffered solutions, emulsifiers, isotonic saline, and pyrogen-free water. Preservatives and other additives can also be present. For example, antimicrobial, antioxidant, chelating agents, and inert gases can be added (see, generally, Remington's Pharmaceutical Sciences, 16th Edition, Mack, (1980)).

The concentration of cystinase in the formulation may vary from about 0.01-100 wt. %.

Those of skill in the art will readily appreciate that dose levels can vary as a function of the specific therapeutic agents, the severity of the symptoms and the susceptibility of the subject to side effects. Preferred dosages for a given therapeutic agent are readily determinable by those of skill in the art by a variety of means. A preferred means is to measure the physiological potency of a given therapeutic agent.

In a preferred embodiment of the present invention, the cystinase is contacted directly with cystine stones in the patient using known methods for perfusing liquids into enclosed cavities such as hollow ducts, organs or even arterial systems of a patient. Such methods include but are not limited to percutaneous catheter placement, endoscopic retrograde biliary catheter placement, or placement of a catheter in a localized area by surgical or nonsurgical means. Following catheter placement, the cystinase solution is flushed through the catheter and into the cavity where it contacts the cystine stone. The solution and dissolved portions of cystine stone are then removed through the catheter and fresh solution is perfused into the cavity. This perfusing technique is typically accomplished using a syringe or pumping system.

In practicing the methods of the present invention, lengths of time which are sufficient to dissolve and/or deaggregate the cystine stone or stones vary and can depend upon a number of factors, including the number, composition and size of the stone(s). Furthermore, in vivo applications require time considerations which are different from the times required for in vitro applications. Clinically, the contact dissolution of stones using perfusion techniques can require more than one treatment procedure with each treatment procedure requiring up to about 8 hours. Treatment procedures can extend over a period of several days, if needed.

### EXAMPLES

### Example 1

### Characterization of Cystathionine-β-Lyase as a Cystinase Enzyme

The enzyme cystathionine β-lyase, has been isolated from E. coli bacteria and catalyzes degradation of L-cystathionine to L-homocysteine in the biosynthesis of L-methionine. (FIG. 1). This same enzyme has also been reported to catalyze the conversion of L-cystine into pyruvate and thiocysteine as shown in FIG. 2 (Uren, 1987).

Cystathionine β-lyase was isolated in crude form from E. coli using standard methods. We showed that this enzyme functions as a very effective cystinase, catalyzing the conversion of L-cystine to pyruvate and thiocysteine rapidly and in high yield. We have further tested cystathionine β-lyase against other amino acids and showed that it is highly selective for L-cystine; none of the 19 other naturally-occurring amino acids react, nor does glutathione. The enzyme is not significantly inhibited by pyruvate or other 2-ketoacids that might be present in urine. In addition to L-cystine, only its natural substrate, L-cystathionine, and two relatively obscure compounds (lanthionine, an unusual amino acid found in some bacteria and in wool hydrolysates, and djenkolic acid, a chemical adduct of 2 cysteine molecules and formaldehyde) have been shown to react.

Cystathionine concentrations in urine are in the micromolar range, typically more than 100-fold lower than for cystine in the urine of cystinurics. Lanthionine and djenkolic acid would not be expected to be present at all. Given the high selectivity of cystathionine β-lyase from E. coli for L-cystine, particularly in the presence of other urine components, this enzyme is particularly well-suited for the dissolution of cystine stones and the lowering of cystine concentrations in urine.

### Example 2

### Cloning and Expression of Cystathionine β-Lyase

Cystathionine β-lyase, encoded by the metC gene, was cloned from E. coli ATCC 37384 using PCR methods based on the known sequence of the gene. Based on the published DNA sequence for the gene from E. coli, two primers were synthesized, incorporating an extra BamHI site at the 5′ ends. The BamHI sites and the start codon are underlined for clarity:

Primer 1:
5′-TAC TCA GGA TCC ATG GCG GAC AAA AAG CTT GAT ACT
CAA CTG G-3′
Primer 2:
5′-GCG TGA GGA TCC TTA TAC AAT TCG CGC AAA ACC
GGC-3′

After BamHI treatment, the amplification product was isolated and subcloned into a linearized BamHI expression vector, and the resulting plasmid was transformed into an E. coli host strain for expression.

### Example 3

### Expression of metC in a Microbial Host Strain to Produce Cystathionine β-Lyase

The cystathionine β-lyase gene was isolated from and expressed in E. coli grown in 400 mL TB media containing 100 mg/μL ampicillin at 30° C. and induced with 0.1 mM isopropyl-β-D-thiogalactopyranoside (IPTG). Cells were harvested and mechanically lysed and the enzyme purified with an ammonium sulfate fractionation, as described below.

Cystathionine β-lyase, encoded by the metC gene, was cloned from E. coli ATCC 37384 using PCR methods based on the published DNA sequence of the gene (Uren, 1987), as in Example 2. We expressed this gene in pET15b/E. coli BL21(DE3) cells. The recombinant E. coli was cultivated in a 15 liter fermentor, following inoculation from shake flasks grown overnight containing 1.2 L of a chemically defined media containing 0.5% glucose and 0.1 mg/mL ampicillin. The glucose limited fed-batch fermentation was carried out with oxygen control at approximately 32° C. Once the optical density of the culture reached an appropriate level (approximately 100), expression of cystathionine β-lyase was induced by the addition of IPTG at a concentration of 0.2 mM. Growth continued for an additional 10 hours, and the cells were harvested by centrifugation at 13,000 g and washed with 50 mM potassium phosphate buffer, pH 7.5, containing 1 mM dithiothreitol (DTT) and 0.2 mM pyridoxal 5-phosphate (PLP). Approximately 5 kilograms of wet cell paste containing 600 g of protein was produced.

### Example 4

### Isolation and Purification of Cystathionine β-Lyase

Approximately 100 g of cell paste was lysed and used for enzyme purification; the remaining cell paste was stored at −20° C. The 100 g of cell paste was resuspended in lysis buffer (50 mM potassium phosphate pH 7.5, 1 mM EDTA, 0.5 mM DTT, 0.2 mM PLP) so that the volume of the suspension was 500 mL (4 mL/g cells). The cell suspension was passed through a homogenizer to lyse the cells and the cellular debris removed by centrifugation (11,500 RPM, 45 min). The remaining soluble enzyme solution was purified with a 30-60% ammonium sulfate precipitation and the pellet resuspended in 400 mL lysis buffer. The resuspended ammonium sulfate pellet was desalted and concentrated using a tangential flow filtration system with a molecular weight cutoff of 10,000 Da, using 20 mM potassium phosphate, pH 7.5, with 0.5 mM EDTA, 1 mM DTT, 0.1 mM PLP as the buffer, followed by lyophilization of the enzyme. The resulting lyophilized cystathionine β-lyase powder was stored at 4° C.

### Example 5

### Dissolution of Cystine Stones Using Cystathionine β-Lyase

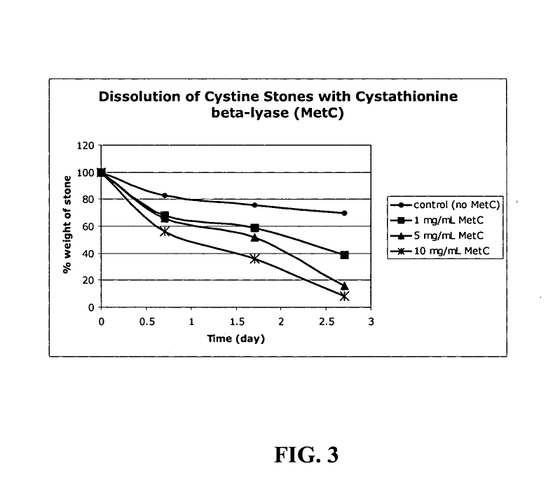

Cystine stones for the dissolution experiments were obtained from a cystinuric volunteer. The cystinuric patient informed us that the stones contained mostly cystine with a small content of calcium oxalate. The cystine stones were taken from a urine sample in which stones had been passed. The dimensions of the stones were approximately 2-3 millimeters in diameter and about 9-15 milligrams in weight. The dissolution experiment was performed in a 6-well plate with four wells containing 4 mL 50mM potassium phosphate, pH 8.0, with 1 mM pyridoxal-5-phosphate (PLP), a cystine stone, and 0, 1, 5 or 10 mg/mL cystathionine β-lyase. The remaining two wells were used as controls with no cystine stone and contained 4 mL 50 mM potassium phosphate, pH 8.0, plus 1 mM PLP and 5 or 10 mg/mL cystathionine β-lyase. The plate was shaken at 30° C. for 3 days. The extent of stone dissolution was determined by first weighing the stones at the start of the experiment and then at each time point the stones were removed, dried under vacuum, weighed again and returned to the plate. The results show that the cystine stones were dissolved and broken up increasingly as the cystathionine β-lyase concentration increased, as shown in FIG. 3.

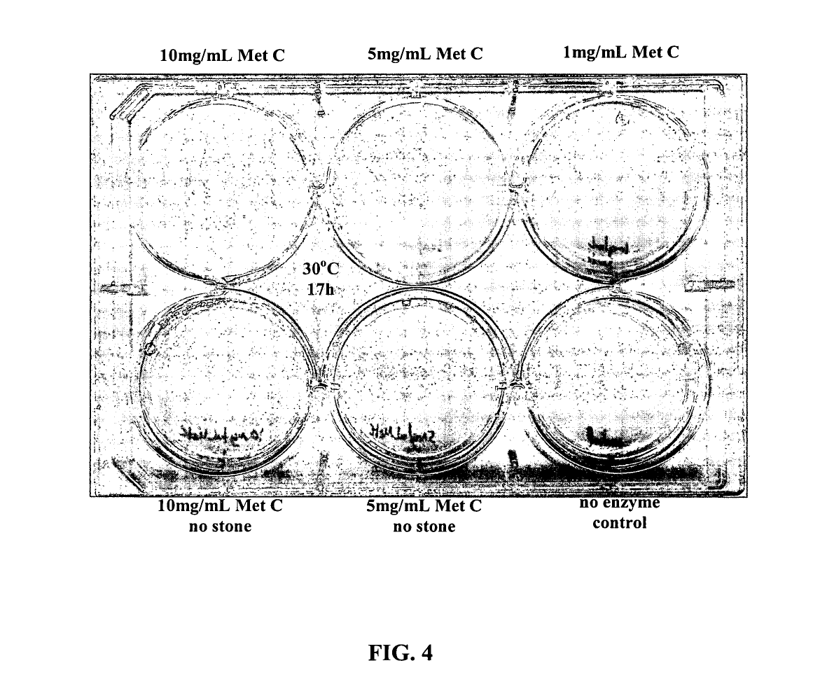

The control without enzyme displayed some background weight reduction of the cystine stone, likely due to a small amount of dissolution required to establish saturation of the solution with cystine. However, a far greater reduction in stone weight was seen in the wells containing cystathionine β-lyase. Scans of the 6-well plate were taken at the various time points and are shown in FIG. 4.

After gentle shaking at 30° C. for 17 hours, the stones were removed and weighed and replaced into the enzyme-containing solution in the plate. The odor of hydrogen sulfide, which is a by-product of the reaction of cystine with cystathionine β-lyase, was also detectable, further suggesting breakdown of the stones by the enzyme. The wells containing cystine stones and 5 and 10 mg/mL cystathionine β-lyase had become cloudy. This cloudiness was most likely due to cystine being released from the stone, because the wells containing the same concentration of cystathionine β-lyase but no stones were not cloudy, verifying that the cloudiness was not due to enzyme precipitation. It was also determined using a spectrophotometric assay that the enzyme did not lose any activity after shaking for 17 hours at 30° C.

### Example 6

### Modification of Cystinase with Polyethyleneglycol (Prophetic)

Pegylation has been shown to stabilize and reduce the immunogenicity of proteins used as pharmaceutical products. The utilization of enzymes as therapeutic agents often requires the covalent attachment of polyethylene glycol (PEG) to the enzyme, referred to as “pegylation”. Pegylation makes an enzyme an improved therapeutic because it provides the enzyme with increased solubility, reduced antigenicity and toxicity, and protection from proteolytic degradation. The use of pegylation has resulted in the FDA approval of several biopharmaceuticals, such as PEG-adenosine deaminase (Adagen) for the treatment of severe combined immunodeficiency syndrome, PEG-asparaginase (Oncaspar) for the treatment of acute lymphoblastic leukemia and PEG-interferon-a2b (PegIntron) for the treatment of hepatitis C. As a further embodiment of this invention, cystinase is modified by the covalent attachment of one or more polyethylene glycol chains (pegylated) prior to administration.

### Example 7

### Entrapping Cystinase in a Polymeric Matrix (Prophetic)

Polymers such a polylalkanoic acid have been used to entrap materials such as enzymes. Cystinase is entrapped in a polymer comprising at least about 50% polylactic acid to create a cystinase complex that has increased stability and slow release characteristics.

### Example 8

### Administering Cystinase via a Catheter (Prophetic)

In order to deliver a therapeutically effective amount of cystinase effectively to the stone to promote stone dissolution, a pharmaceutical composition comprising cystinase is delivered through a catheter into the kidney area. The pharmaceutical composition is delivered as a liquid or a gel or as a finely dispersed solid, as needed.

### REFERENCES

Bailon and Berthold, 1998, P. Bailon, W. Berthold, Pharmaceut. Sci. Technol. Today, 1, 352-356 (1998).
Beaudet et al, 1995, The Metabolic and Molecular basis of Inherited Disease, McGraw-Hill Co., New York, 7^{th} edition, A. L. Beaudet, W. S. Sly, and D. Valle eds. (1995).
Chow and Streem, 1996, G. K. Chow, S. B. Streem, J. Urol., 156, 1576-1578 (1996).
Davis and Metzler, 1972, Leodis Davis and David E. Metzler in The Enzymes, volume VII, pp. 51-52, Paul Boyer, editor, Academic Press, New York, (1972).
Dwivedi et al, 1982, Chandra M. Dwivedi, Richard C. Ragin, and Jack R. Uren, Biochemistry, 21, 3064-3069 (1982).
Gentry-Weeks et al., 1993, C. R. Gentry-Weeks, J. M. Keith, J. Thompson, J. Biol. Chem., 268, 7298-7314 (1993).
Harris and Chess, 2003, J. M. Harris, R. B. Chess, Nat. Rev. Drug Discov., 2, 214-221 (2003).
Heimbach et al., 2000, D. Heimbach, D. Jacobs, S. C. Muller, A. Hesse, Urology, 55, 17-21 (2000).
Kurfurst, 1992, M. M. Kurfurst, Anal. Biochem., 200, 244-248 (1992).
Ng and Streem, 2001, C. S. Ng, S. B. Streem, Curr. Opin. Urol., 11, 353-358 (2001).
Pearle and Preminger, 2005, M. S. Pearle, G. M. Preminger, Urolithiasis and Endourology Update Conference, Tucson, Ariz., Feb. 6, 2005.
Roberts et al., 2002, M. J. Roberts, M. D. Bentley, J. M. Harris, Adv. Drug Deliv. Rev., 54, 459-476 (2002).
Rohrbach et al, 1973, M. S. Rohrbach, B. A. Humphries, F. J. Yost, Jr., W. G. Rhods, S. Boatman, and J. H. Harrison, Anal. Biochem. 52, 127 (1973).
Rozzell, 1992, “Immobilization of Enzymes: Techniques and Applications”: J. David Rozzell in Biocatalytic Production of Amino Acids and Derivatives: New Developments and Process Considerations, D. Rozzell and F. Wagner, editors, Hanser Publishers (1992).
Sakhaee and Sutton, 1996, Khashayar Sakhaee and Roger A. L. Sutton in Kidney Stones: Medical and Surgical Management Chapter 46, F. L. Coe, M. J. Favus, C. Y. C. Pak, J. H. Parks, and G. M. Preminger, editors, Lippincott-Raven Publishers, Philadelphia (1996).
Skursky et al, 1979, L. Skursky, J. Kovar, and Milada Stachova, Analytical Biochemistry, 99, 65-71 (1979).
Uren, 1987, Jack R. Uren, Methods in Enzymology, 84, 483-486 (1987).

While this invention has been described in detail with reference to a certain preferred embodiments, it should be appreciated that the present invention is not limited to those precise embodiments. Rather, in view of the present disclosure which describes the current best mode for practicing the invention, many modifications and variations would present themselves to those of skill in the art without departing from the scope and spirit of this invention. For example, although some of the examples contained herein describe the action of the cystinase cystathionine β-lyase, other cystinases may be used in practice of the present invention, as will be appreciated by one of skill in the art.

In particular, it is to be understood that this invention is not limited to the particular methodology, protocols, cell lines, animal species or genera, constructs, and reagents described as such may vary, as will be appreciated by one of skill in the art. The scope of the invention is, therefore, indicated by the following claims rather than by the foregoing description. All changes, modifications, and variations coming within the meaning and range of equivalency of the claims are to be considered within their scope.

1. A method for dissolving cystine stones, comprising the step of administering to a patient a therapeutically effective amount of a cystinase.

2. (canceled)

3. The method of claim 1, wherein the cystinase is cystathionine β-lyase.

4. The method of claim 1, wherein the cystinase is selected from the group consisting of cystathionine-gamma-lyase, cystine transaminase, cystine transhydrogenase, cystine reductase, cystathionine β-lyase, and mixtures thereof.

5. The method of claim 1, wherein the cystinase is covalently attached to a polymer.

6. The method of claim 5, wherein the polymer is polyethylene glycol.

7. The method of claim 1 wherein the cystinase is entrapped in a polymeric material.

8. The method of claim 7 wherein the polymeric material comprises at least about 50% of a polyalkanoic acid.

9. The method of claim 8 wherein the polyalkanoic acid is polylactic acid.

10. The method of claim 1 wherein the cystinase is administered via a catheter into the kidney of the patient.

11. A method for the treatment of cystinuria comprising administering to a patient in need thereof a therapeutically effective amount of a cystinase.

12. The method of claim 11, wherein the cystinase is cystathionine β-lyase.

13. The method of claim 11, wherein the cystinase is selected from the group consisting of cystathionine-gamma-lyase, cystine transaminase, cystine transhydrogenase, cystine reductase, cystathionine β-lyase, and mixtures thereof.

14. The method of claim 11, wherein the cystinase is covalently attached to a polymer.

15. The method of claim 14, wherein the polymer is polyethylene glycol.

16. The method of claim 11 wherein the cystinase is entrapped in a polymeric material.

17. The method of claim 16 wherein the polymeric material comprises at least about 50% of a polvalkanoic acid.

18. The method of claim 17 wherein the polyalkanoic acid is polylactic acid.

19. The method of claim 11 wherein the cystinase is administered via a catheter into the kidney of the patient.

20. A method for lowering the concentration of cystine in a patient's urine, comprising the step of administering a therapeutically effective amount of a cystinase to the patient.

21. The method of claim 20, wherein the cystinase is cystathionine β-lyase.

22. The method of claim 20, wherein the cystinase is selected from the group consisting of cystathionine-gamma-lyase, cystine transaminase, cystine transhydrogenase, cystine reductase, cystathionine β-lyase, and mixtures thereof.

23. The method of claim 20, wherein the cystinase is covalently attached to a polymer.

24. The method of claim 23, wherein the polymer is polyethylene glycol.

25. The method of claim 20 wherein the cystinase is entrapped in a polymeric material.

26. The method of claim 25 wherein the polymeric material comprises at least about 50% of a polyalkanoic acid.

27. The method of claim 26 wherein the polyalkanoic acid is polylactic acid.

28. The method of claim 20 wherein the cystinase is administered via a catheter into the kidney of the patient.
